(19) United States
(12) Patent Application Publication
(10) Pub. No.: US 2006/0035221 A1
(43) Pub. Date: Feb. 16, 2006

(54) Use of the glycine n-acyl transferase (gnat) for the diagnosis and therapy of inflammatory diseases and sepsis

(75) Inventors: Andreas Bergmann, Berlin (DE); Joachim Struck, Berlin (DE); Monika Uhlein, Berlin (DE)

Correspondence Address: MAYER, BROWN, ROWE & MAW LLP, 1909 K STREET, N.W., WASHINGTON, DC, 20006, US

(73) Assignee: B.R.A.H.M.S Aktiengesellschaft, Hennigsdorf (DE)

(21) Appl. No.: 10/497,679

(22) PCT Filed: Nov. 29, 2002
(86) PCT No.: PCT/EP02/13524

§ 371 Date: Feb. 1, 2005

(30) Foreign Application Priority Data
Dec. 4, 2001 (EP) 01128852.9

(51) Int. Cl. C12Q 1/68; C12Q 1/48

(52) U.S. Cl. 435/6; 435/15

(57) ABSTRACT

Uses of glycine N-acyltransferase (GNAT) from body fluids or body tissues in human and veterinary medicine as a marker peptide for diagnosis, for prognosis and for monitoring the course of inflammations and infections and/or as a target for therapeutically influencing the course of inflammations and/or infections.

The present invention relates to novel uses of the enzyme glycine N-acyltransferase (always abbreviated to GNAT below) for the medical diagnosis and therapy of inflammatory diseases and sepsis. It is based on the detection for the first time of greatly increased concentrations of GNAT in liver tissue of primates in which a sepsis or systemic inflammation had been produced experimentally by toxin administrations.

The present invention has its origin in intensive research work by the Applicant in relation to further improvements of the diagnosis and therapy of inflammations and infections, in particular of inflammations of infectious aetiology and sepsis.

Inflammations are defined very generally as certain physiological reactions of an organism to different types of external effects, such as, for example, injuries, burns, allergens, infections by microorganisms, such as bacteria and fungi and viruses, to foreign tissues which trigger rejection reactions, or to certain inflammatory endogenous conditions of the body, for example in autoimmune diseases and cancer. Inflammations may occur as harmless, localized reactions of the body but are also typical features of numerous serious chronic and acute diseases of individual tissues, organs, organ parts and tissue parts.

Local inflammations are generally part of the healthy immune reaction of the body to harmful effects and hence part of the life-preserving defence mechanism of the body. If, however, inflammations are part of a misdirected reaction of the body to certain endogenous processes, such as, for example, in autoimmune diseases, and/or are of a chronic nature, or if they achieve a systemic extent, as in the case of systemic inflammatory response syndrome (SIRS) or in the case of a severe sepsis caused by infection, the physiological processes typical of inflammatory reactions go out of control and become the actual, frequently life-threatening pathological process.

It is now known that the origin and the course of inflammatory processes are controlled by a considerable number of substances which are predominantly of a protein or peptide nature, or are accompanied by the occurrence of certain biomolecules for a more or less limited time. The endogenous substances involved in inflammatory reactions include in particular those which may be counted among the cytokines, mediators, vasoactive substances, acute phase proteins and/or hormonal regulators. The inflammatory reaction is a complex physiological reaction in which both endogenous substances activating the inflammatory process (e.g. TNF-α) and deactivating substances (e.g. Interleukin-10) are involved.

In systemic inflammations, as in the case of a sepsis or of septic shock, the inflammation-specific reaction cascades spread in an uncontrolled manner over the whole body and become life-threatening in the context of an excessive immune response. Regarding the current knowledge about the occurrence and the possible role of individual groups of endogenous inflammation-specific substances, reference is made, for example, to A. Beishuizen et al., “Endogenous Mediators in Sepsis and Septic Shock”, Advances in Clinical Chemistry, Vol. 33, 1999, 55-131; and C. Gabay et al., “Acute Phase Proteins and Other Systemic Responses to Inflammation”, The New England Journal of Medicine, Vol. 340, No. 6, 1999, 448-454. Since the understanding of sepsis and related systemic inflammatory diseases, and hence also the recognized definitions, have changed in recent years, reference is also made to K. Reinhart et al., “Sepsis und septischer Schock” [Sepsis and septic shock] in: Intensivmedizin, Georg Thieme Verlag, Stuttgart, New York, 2001, 756-760, where a modern definition of sepsis is given. In the context of the present invention, the terms sepsis and inflammatory diseases used are based on the definitions given in the stated three literature references.

Whereas at least in Europe the systemic bacterial infection detectable by a positive blood culture long characterized the term sepsis, sepsis is now primarily understood as being systemic inflammation which is caused by infection but, as a pathological process, has great similarities to systemic inflammations which are triggered by other causes. Said transformation in the understanding of sepsis has resulted in changes in the diagnostic approaches. Thus, the direct detection of bacterial pathogens was replaced or supplemented by complex monitoring of physiological parameters and, more recently, in particular by the detection of certain endogenous substances involved in the sepsis process or in the inflammatory process, i.e. specific “biomarkers”.

Of the large number of mediators and acute phase proteins which are known to be involved in an inflammatory process, the ones which are suitable for diagnostic purposes are in particular those whose occurrence is very specific for inflammatory diseases or certain phases of inflammatory diseases, whose concentrations change in a dramatic and diagnostically significant manner and which moreover have the stabilities required for routine determinations and reach high concentration values. For diagnostic purposes, the reliable correlation of pathological process (inflammation, sepsis) with the respective biomarker is of primary importance, without there being any need to know its role in the complex cascade of the endogenous substances involved in the inflammatory process.

Such an endogenous substance particularly suitable as a sepsis biomarker is procalcitonin. Procalcitonin is a prohormone whose serum concentrations reach very high values under the conditions of a systemic inflammation of infectious aetiology (sepsis), whereas it is virtually undetectable in healthy persons. High values of procalcitonin are also reached in a relatively early stage of sepsis so that the determination of procalcitonin is also suitable for early diagnosis of a sepsis or for early distinguishing of a sepsis caused by infection from severe inflammations which have other causes. The determination of procalcitonin as a sepsis marker is the subject of the publication by M. Assicot et al., “High serum procalcitonin concentrations in patients with sepsis and infection”, The Lancet, Vol. 341, No. 8844, 1993, 515-518; and the patents DE 42 27 545 C2 and EP 0 656 121 B1 and U.S. Pat. No. 5,639,617. Reference is hereby made to said patents and to early literature references mentioned in said publication for supplementing-the present description. In recent years, the number of publications on the subject of procalcitonin has greatly increased. Reference is therefore also made to W. Karzai et al., “Procalcitonin—A New Indicator of the Systemic Response to Severe Infection”, Infection, Vol. 25, 1997, 329-334; and M. Oczenski et al., “Procalcitonin: a new parameter for the diagnosis of bacterial infection in the peri-operative period”, European Journal of Anaesthesiology 1998, 15, 202-209; and furthermore H. Redl et al., “Procalcitonin release patterns in a baboon model of trauma and sepsis: Relationship to cytokines and neopterin”, Crit Care Med 2000, Vol. 28, No. 11, 3659-3663; and H. Redl et al., “Non-Human Primate Models of Sepsis”, in: Sepsis 1998; 2:243-253; and the further literature references cited therein, as typical of recent published reviews.

The availability of the sepsis marker procalcitonin has given considerable impetus to sepsis research, and intensive efforts are now being made to find further biomarkers which can supplement the procalcitonin determination and/or are capable of providing additional information for purposes of fine diagnosis or differential diagnosis. The search for potential new sepsis biomarkers is, however, complicated by the fact that frequently very little or nothing is known about the exact function or about the exact reasons for the occurrence of certain endogenous substances which are involved in the inflammatory or septic process.

The results of the experimental testing of a fruitful purely hypothetical approach to the determination of further potential sepsis markers are to be found in DE 198 47 690 A1 and WO 00/22439. There, it is shown that, in the case of sepsis, not only is the concentration of the prohormone procalcitonin increased but also significantly increased concentrations can be observed for other substances which may be included among the peptide prohormones. While the phenomenon described is well documented, the causes of the increase in the concentrations of prohormones in sepsis are still substantially unexplained.

In the present Application, a result of another fruitful, purely experimental approach in the search for further inflammation- or sepsis-specific biomolecules is reported. These experimental investigations, too, originate in the determination of procalcitonin in relation to systemic inflammatory reactions of infectious aetiology. Thus, it had been observed at a very early stage that the procalcitonin is evidently not formed in the same manner in sepsis as when it is a precursor for the hormone calcitonin. Thus, high procalcitonin levels were also observed in patients whose thyroid had been removed. The thyroid therefore cannot be the organ in which procalcitonin is formed or secreted during sepsis. In the publications by H. Redl et al., “Procalcitonin release patterns in a baboon model of trauma and sepsis: Relationship to cytokines and neopterin”, Crit Care Med 2000, Vol. 28, No. 11, 3659-3663; and H. Redl et al., “Non-Human Primate Models of Sepsis”, Sepsis 1998; 2:243-253, the results of experimental investigations which are said to be intended for explaining the formation of procalcitonin in sepsis are reported. In said work, an artificial sepsis is produced by endotoxin administration to primates (baboons), and the experimentally produced states in which the highest procalcitonin concentrations in the blood are reached are determined. A further development of the experimental animal model described in said work serves, in the context of the present Application, for determining novel endogenous sepsis-specific biomarkers of a peptic or protein nature, the occurrence of which is characteristic of sepsis or certain forms of sepsis and which therefore permit a specific diagnosis of sepsis. The primate model was chosen because of the very great similarity of the physiology of primates and humans and the high cross-reactivity with many therapeutic and diagnostic human reagents.

Since the endogenous substances formed during inflammations are part of the complex reaction cascade of the body, not only are such substances also of diagnostic interest but attempts are currently also being made, with considerable effort, to intervene therapeutically in the inflammatory process by influencing the formation and/or the concentration of individual substances of this type, in order to stop as early as possible the systemic spread of the inflammation, which spread is observed, for example, during sepsis. In this context, endogenous substances which have been shown to be involved in the inflammatory process are also to be regarded as potential therapeutic targets. Attempts based on certain mediators of the inflammatory process and intended to have a positive therapeutic influence on said process are described, for example, in E. A. Panacek, “Anti-TNF strategies”, Journal für Anästhesie und Intensivbehandlung; No. 2, 2001, 4-5; T. Calandra et al., “Protection from septic shock by neutralization of macrophage migration inhibitory factor”, Nature Medicine, Vol. 6., No. 2, 2000, 164-170; or K. Garber, “Protein C may be sepsis solution”, Nature Biotechnology, Vol. 18, 2000, 917-918. These therapeutic approaches are intended to lower the concentrations of inflammation-promoting substances or to inhibit the formation of such substances, and to do so in particular with the use of specific antibodies (against TNF-α or MIF; cf. E. A. Panacek, “Anti-TNF strategies”, Journal für Anasthesie und Intensivbehandlung; No. 2, 2001, 4-5; T. Calandra et al., “Protection from septic shock by neutralization of macrophage migration inhibitory factor”, Nature Medicine, Vol. 6, No. 2, 2000, 164-170) or to increase the concentration of endogenous substances which have an inhibitory effect in the inflammation cascade (Protein C; K. Garber, “Protein C may be sepsis solution”, Nature Biotechnology, Vol. 18, 2000, 917-918). The last-mentioned publication gives an overview of such attempts to have a therapeutic influence on the inflammatory process by influencing selected endogenous target molecules, which attempts have unfortunately generally met with little success to date. In view of the rather disappointing therapeutic approaches to date, there is great interest in identifying further endogenous biomolecules which are as inflammation- or sepsis-specific as possible and which, as therapeutic targets, also open up new prospects for success in fighting inflammation.

The present invention is based on the fact that the enzyme glycine N-acyltransferase (GNAT) is detectable in considerable concentrations in primates and humans during inflammations caused by infection, in contrast to healthy persons in whom it is not found or is found only in concentrations at the analytical limit of detection, which makes GNAT suitable both for inflammation diagnosis/sepsis diagnosis and as a novel therapeutic target.

The uses in diagnosis and therapy which result from the first detected occurrences of GNAT in the experimental simulation of inflammations or sepsis are claimed in general form in claim 1.

Claims 2 to 9 relate to diagnostic uses and methods.

Claim 10 relates in general form to the novel potential therapeutic uses, in particular in the area of the therapy of inflammations and infections, including sepsis, and claims 11 to 13 relate to pharmaceutical compositions intended for therapeutic applications which are aimed at influencing the physiological GNAT concentrations.

As will be explained in more detail below in the experimental section, the invention is based on the fact that, after experimental triggering of an artificial sepsis in baboons by endotoxin administration (LPS from Salmonella Typhimurium) and working-up of liver tissue of the treated animals by 2D gel electrophoresis, a peptide or protein product identifiable only in the treated animals is found. This specific product was isolated from the electrophoresis gel and investigated in a manner known per se by a sequence analysis of the N-terminus, which led to the identification of a partial peptide of 8 amino acids (SEQ ID NO:1) and, on the other hand, by mass spectrometry. It was found that the partial peptide of the N-terminus occurs in a human cDNA database in an EST (GenBank Accession No. AB013093) which on translation gives the polypeptide with the amino acid sequence according to SEQ ID NO:2. On the basis of a 76% identity of said amino acid sequence with the known sequence of bovine GNAT (GenBank Accession No. AF045032), it was possible to identify the peptide found in the manner described as human GNAT.

In the mass spectrometric analysis of the protein spot of the protein from baboon liver by tandem mass spectrometry, numerous short peptide sequences were found which surprisingly did not occur in identical form in any sequence from the human genome. After identification of the human GNAT sequence (SEQ ID NO:2), it was however possible to assign the sequences identified by means of mass spectrometry to corresponding partial sequences of human GNAT. On comparison of the identified sequences from the baboon protein with the sequence of human GNAT, it was found that deletions or an exchange of one or more amino acids were or was detectable in the case of the partial sequences identified, all of which were in the range of the amino acids 140 to 280 of human GNAT (SEQ ID NO:2). Evidently, the GNAT peptides are among the peptides which vary greatly beyond the family and generic limits, i.e. are poorly conserved, this also being indicated by the considerable differences between the human GNAT found and the known bovine GNAT.

On the basis of the identity of the sequence of the N-terminus, found by sequencing, and the homology of the mass spectrometrically identified baboon partial sequences and human partial sequences, which is high in spite of the differences described, the identification of the human equivalent of the isolated protein spot as GNAT is to be regarded as unambiguous according to recognized criteria.

When, in the present Application, the peptide intended for diagnostic purposes or proposed for therapeutic purposes is referred to as “GNAT”, this does not mean that such a GNAT must be 100% identical to the sequence according to SEQ ID NO:2. Rather, in the present Application, GNAT is defined as a peptide which, in the physiologically occurring form, has a peptide sequence according to SEQ ID NO:1 at its N-terminus and has a high homology, i.e. preferably of more than 60%, more preferably 80%, with the sequence of human GNAT according to SEQ ID NO:2. The term GNAT also covers partial peptides of a peptide as defined above, which can be used in particular in the preparation of reagents, for example selective antibodies, for the GNAT determination or the preparation of assays for GNAT determination in biological samples. Those sequences which are obtained after deletion of one or more amino acids or short peptide sequences from the peptide according to SEQ ID NO:2 are also to be regarded as GNAT or partial sequences thereof. Furthermore, partial sequences (fragments) suitable for diagnostic and/or therapeutic purposes are in particular those which comprise a sequence of at least three amino acids, preferably at least 6 amino acids, of the peptide SEQ ID NO:2.

For particular diagnostic or therapeutic purposes, the GNAT peptides according to the invention may also be mammalian peptides, which should have at least 60% homology with the peptide having SEQ ID NO:1 and are used, for example, for diagnostic purposes or in veterinary medicine.

The identification of the protein found only after triggering of sepsis or of inflammation in baboon liver tissue as GNAT is to be regarded as being unambiguous according to recognized criteria and is of considerable scientific, diagnostic and therapeutic interest. Various forms of GNAT have long been known for enzymatic tissue activity. To our knowledge, the cDNA sequence present in the database has not yet been assigned to human GNAT. The GNAT activity is a subject of investigations with a primarily scientific aim. It is possible to mention here, for example, publications by Margaret O. James et al., Biochem. J. (1978) 172, 285-291; M. Kelley et al., J. Biochem. Toxicology, Vol. 8, No. 2, pp. 63-69 (1993); Yogesh R. Marwal et al., Biochem. Biophys. Res. Comm. Vol. 205, No. 2, 1994, pp. 1373-1379; D. J. Merkler et al., Archives of Biochemistry and Biophysics, Vol. 330, No. 2, pp. 430-434, 1996; Yogesh R. Marwal et al., J Pediatr 1997, 130, 1003-1007; Francois H. van der Westhuizen et al., J. Biochem. Toxicol 14:102-109, 2000. In said papers, GNAT is discussed primarily as an enzyme which plays an important role in the detoxification of the organism of mammals by the peptide-binding conjugation of endogenous and exogenous carboxylic acids. GNAT has played no role to date in medical diagnosis and therapy.

With the present invention, it is desired on the one hand, according to claim 1, to protect the uses mentioned therein of a peptide which is referred to as GNAT and, according to the above-mentioned definition, is defined primarily by the presence of the partial sequence of 8 amino acids according to SEQ ID NO:1 and is thus distinguishable from all other known human peptides and proteins, without there being any intention for there to be restrictions with respect to the length and nature of further amino acid sequences.

On the basis of the sequence now known and of the physiological role of human GNAT in combination with the findings concerning its increased formation in the case of inflammations and sepsis, human GNAT or parts thereof can be prepared by synthesis or genetic engineering as recombination products for diagnostic and/or therapeutic purposes by methods which are now part of the prior art.

Furthermore, GNAT peptides can be used by known methods of the modern prior art also for the production of specific polyclonal and in particular monoclonal antibodies which are suitable as aids for the diagnostic determination of the peptides according to the invention and/or as potential therapeutic agents. The production of suitable monoclonal antibodies against known partial peptide sequences is now part of the general prior art.

In the determination of GNAT or selected partial peptides thereof, it is possible to proceed in principle as described, for example for the selective procalcitonin determination, in P. P. Ghillani et al., “Monoclonal antipeptide antibodies as tools to dissect closely related gene products”, The Journal of Immunology, Vol. 141, No. 9, 1988, 3156-3163; and P. P. Ghillani et al., “Identification and Measurement of Calcitonin Precursors in Serum of Patients with Malignant Diseases”, Cancer Research, Vol. 49, No. 23, 1989, 6845-6851, the immunization techniques described there, which represent a possibility for obtaining monoclonal antibodies also against partial sequences of GNAT, hereby being incorporated by reference. Variations of the techniques described and/or further immunization techniques can be adopted by a person skilled in the art from relevant standard works and publications and applied in context.

Furthermore, the production of GNAT antibodies using techniques of direct genetic immunization with DNA should also be expressly mentioned. It is furthermore within the scope of the present invention to use for the immunization, for example, a cDNA of the desired GNAT or its partial peptides, since it has been found in the past that the spectrum of obtainable antibodies can be extended by said immunization techniques. GNAT according to SEQ ID NO:2 or partial peptides thereof, for example those which contain the partial sequence SEQ ID NO:1 and/or other partial sequences, can, on the basis of the available results, serve as specific marker peptides (biomarkers) for diagnosis and for monitoring the course of inflammations and infections (in particular of systemic infections of the sepsis type). As in the case of the determination of procalcitonin, the determination of GNAT can be effected by the use of a method for early differential diagnosis and for detection and for assessment of the severity and for the therapy-accompanying assessment of the course of sepsis and infections, in such a method the content of GNAT or of a partial peptide thereof being determined in a sample of biological fluid or of a tissue of a patient and conclusions being drawn from the established presence and/or amount of the peptide determined with regard to the presence of an inflammation, of a severe infection or of a sepsis and the result obtained being correlated with the severity of the sepsis and the possibilities for treatment and/or the prospects of the treatment being assessed.

Instead of the determination of GNAT or of its fragments or optionally posttranslationally modified forms thereof, the determination of the associated mRNA is also possible for diagnostic purposes. For diagnostic purposes, the GNAT determination can also be carried out indirectly as a determination of its enzymatic activity in an inflamed organ or tissue or in a biological fluid.

It is furthermore possible to carry out the determination of GNAT as a prognosis marker and marker for monitoring the course of inflammations, in particular systemic inflammations, and sepsis as part of a combination measurement with other markers.

In addition to a combination with a procalcitonin measurement, a combination of the measurement of GNAT with the determination of other markers for sepsis and systemic inflammations is particularly suitable, especially with CA 19-9, CA 125, S100B or S100A proteins involved in the regulation of inflammations, or with the determination of the novel sepsis markers inflammin (DE 101 19 804.3) and CHP (DE 101 31 922.3) described in the prior unpublished German Patent Applications of the Applicant which are mentioned below, and/or with the determination of soluble cytokeratin fragments, in particular of the recently found soluble cytokeratin-1 fragments (sCYlF; DE 101 30 985.6) and of the known tumour marker CYFRA-21 or TPS and/or of one or more of the above-mentioned prohormones. A simultaneous determination of the known inflammation parameter C-reactive protein (CRP) can also be provided. On the basis of the novel results described in this Application and in the parallel Applications, a combination with measurements of biomolecules which are known or still to be found and which are suitable as tissue- or organ-specific inflammation markers should also generally be considered for the fine diagnosis of sepsis.

Said prior Applications of the Applicant are hereby incorporated by reference.

GNAT or its fragments or fusion products or the DNA coding therefor can also be used in preventive medicine or therapy. Thus, for example, suitable GNAT fragments can be used for the in vivo production of GNAT-binding antibodies by active immunization by techniques known per se. Those molecules which contain the complete GNAT or suitable partial sequences thereof in posttranslationally modified form, for example in glycosylated or phosphorylated form, or in a form substituted by pharmaceutical excipients, e.g. polyethylene glycol radicals, are also to be regarded as GNAT. GNAT or suitable partial sequences thereof can also serve as a target for therapeutic intervention in the sense that, by means of suitable specific binders for GNAT or partial peptides thereof, GNAT is deactivated intracorporeally or optionally is also eliminated extracorporeally in the context of a “lavage of the blood” or plasmapheresis using suitable immunoadsorbents or solid phases from the circulation of patients, which phases are coated with specific binders for GNAT and can be perfused.

In particular specific antibodies, especially humanized monoclonal antibodies, are suitable for the in vivo deactivation of GNAT. However, the inflammation cascade can also be therapeutically influenced using GNAT itself or GNAT agonists or GNAT antagonists. Such therapeutic interventions are possible in particular when further discoveries relating to the physiological function of GNAT in an inflammatory process have been confirmed. Thus, it cannot at present be ruled out that GNAT plays an important role in the inflammatory process, possibly by a direct or indirect influence on the pathological process by the route of acylation of peptides, proteins, lipids, sugar molecules and other substances.

The discovery and identification of GNAT is described in more detail below, reference being made to the attached sequence listing. In the Figures:

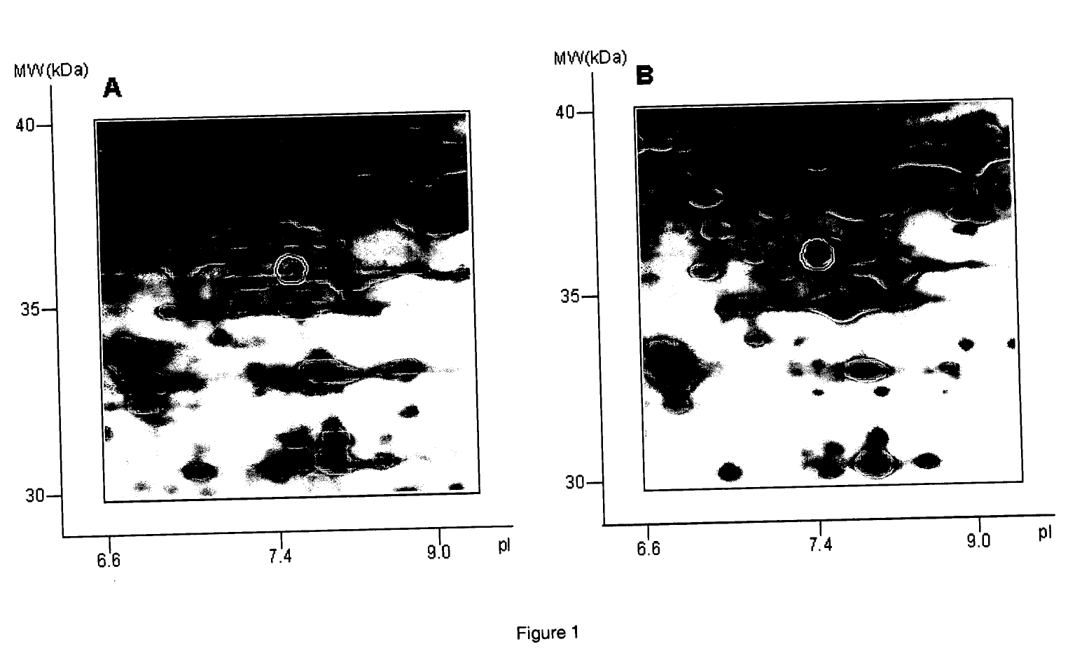

FIG. 1 shows views of 2D electrophoresis gels which permit a comparison of the spot samples of cytoplasmic liver cell protein of a healthy baboon (A) with the liver cell proteins of a baboon 5 h after a sepsis induced by LPS administration (B) . The arrow indicates the position of the sepsis-specific product GNAT according to the invention, which is emphasized in representation (B) by a circle;

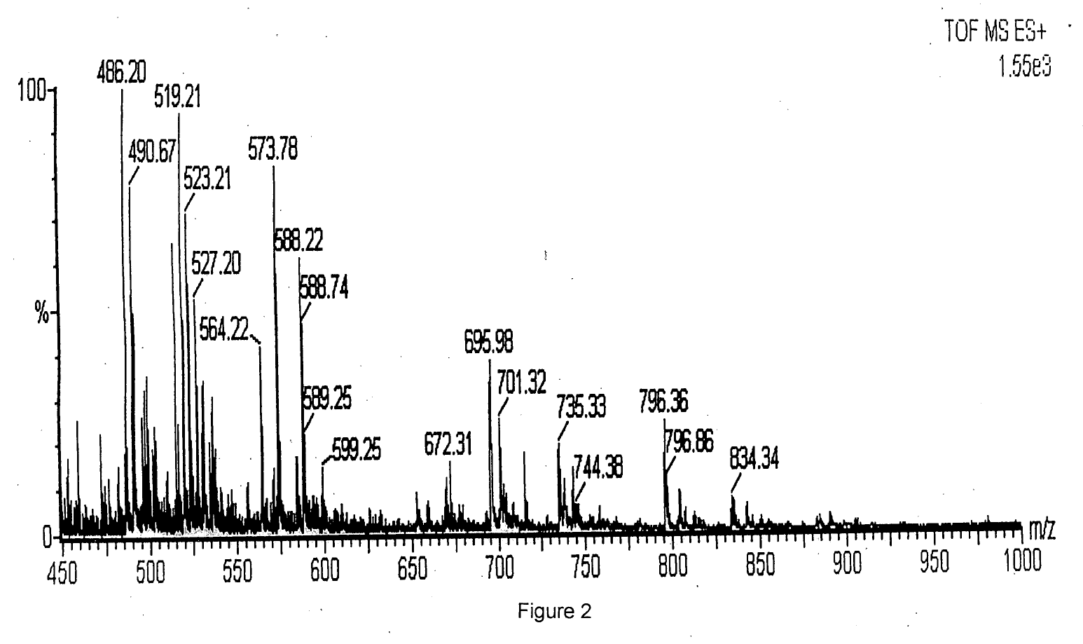

FIG. 2 shows the mass spectrum of the protein spot from the baboon liver (baboon GNAT) after trypsin digestion thereof, said protein spot having been identified by 2D gel electrophoresis.

### 1. Infection Simulation by Endotoxin Administration in an Animal Model (Baboons)

On the basis of the experiments carried out with baboons for the stimulation of procalcitonin secretion by. endotoxin injections (cf. H. Redl et al., “Procalcitonin release patterns in a baboon model of trauma and sepsis: Relationship to cytokines and neopterin”, Crit Care Med 2000, Vol. 28, No. 11, 3659-3663; H. Redl et al., “Non-Human Primate Models of Sepsis”, in: Sepsis 1998; 2:243-253), baboons (male, about 2 years old, weighing from 27 to 29 kg) were each intravenously administered 100 μg of LPS (lipopolysaccharide from Salmonella Typhimurium, source: Sigma) per kg body weight. From 5 to 5.5 h after the injection, the animals were sacrificed by intravenous administration of 10 ml of doletal. Within 60 min of their death, all organs/tissues were dissected and were stabilized by freezing in liquid nitrogen.

During the further processing, 1.5 ml of buffer A (50 mM Tris/HCl, pH 7.1, 100 mM KCl, 20% of glycerol) were added to samples of the individual frozen tissues (1 g) while cooling with nitrogen, and the samples were pulverized in a porcelain mortar to give a powder (cf. J. Klose, “Fractionated Extraction of Total Tissue Proteins from Mouse and Human for 2-D Electrophoresis”, in: Methods in Molecular Biology, Vol. 112: 2-D Proteome Analysis Protocols, Humana Press Inc., Totowa, N.J.). After subsequent centrifuging for 1 hour at 100,000 g and +4° C., the supernatant obtained was recovered and was stored at −80° C. until required for further processing.

Because experiments with the samples obtained as above showed that the largest amount of procalcitonin is found in liver tissue of treated animals, protein extracts from a baboon liver were employed in the search for novel sepsis-specific biomarkers.

### 2. Proteome Analysis Using Cytoplasmic Liver Cell, Proteins of Baboons

Cytoplasmic liver cell protein extracts of, on the one hand, healthy baboons (control) and, on the other hand, baboons which had been injected with LPS were used in a proteome analysis. In the initial analytical 2D gel electrophoresis, liver extract containing 100 μg of protein was stabilized to 9M urea, 70 mM DTT, 2% ampholyte pH 2-4 and then separated by means of analytical 2D gel electrophoresis, as described in J. Klose et al., “Two-dimensional electrophoresis of proteins: An updated protocol and implications for a functional analysis of the genome”, Electrophoresis 1995, 16, 1034-1059. The visualization of the proteins in the 2D electrophoresis gel was effected by means of silver staining (cf. J. Heukeshoven et al., “Improved silver staining procedure for fast staining in Phast-System Development Unit. I. Staining of sodium dodecyl gels”, Electrophoresis 1988, 9, 28-32).

For evaluation, the protein spot patterns of the samples of untreated animals were compared with the protein spot patterns which resulted from liver tissue samples of treated animals. Substances which occurred in no control sample but additionally in all treated animals were selected for further analytical investigations. FIG. 1 shows a comparison of the 2D electrophoresis gels for a control sample (A) and a sample of a treated animal (B), the additional protein spot in (B) corresponding to GNAT, the position of which is singled out by an arrow and a circle.

The novel specific proteins identified in the protein spot pattern of the analytical 2D gel electrophoresis were then prepared by means of preparative 2D gel electrophoresis using 350 μg of protein (once again cf. (10)). In the preparative 2D gel electrophoresis, the staining was effected by means of Coomassie Brilliant Blue G250 (cf. V. Neuhoff et al., “Improved staining of proteins in polyacrylamide gels including isoelectric focusing gels with clear background at nanogram sensitivity using Coomassie Brilliant Blue G-250 and R-250”, Electrophoresis 1988, 9, 255-262).

The protein spot preselected for the further analysis was cut out of the gel.

For the N-terminal sequencing, the separated protein spot is transferred by means of electroblotting to PVDF membranes by the method which was described by P. Matsudaira in: J. Biol. Chem., Vol. 262, No. 21, pp. 10035-10038, 1987. The protein immobilized on PVDF membranes was then end-sequenced by means of automatic Edman degradation using a ProCise® sequencer, equipped with a 140 C microgradient system and a 785 programmable detector (PE-Applied Biosystems), by the manufacturer's methods. A peptide which comprises 8 amino acids and has the sequence shown in SEQ ID NO:1 was identified for the protein spot characterized in FIG. 1B.

Furthermore, the protein spot was trypsin-digested using the method which was described in A. Otto et al., “Identification of human myocardial proteins separated by two-dimensional electrophoresis using an effective sample preparation for mass spectrometry”, Electrophoresis 1996, 17, 1643-1650, and then analyzed by means of mass spectroscopy, in particular with the use of mass spectrometric investigations as described and discussed, for example, in G. Neubauer et al., “Mass spectrometry and EST-database searching allows characterization of the multi-protein spliceosome complex”, in: nature genetics vol. 20, 1998, 46-50; J. Lingner et al., “Reverse Transcriptase Motifs in the Catalytic Subunit of Telomerase”, in: Science, Vol. 276, 1997, 561-567; M. Mann et al., “Use of mass spectrometry-derived data to annotate nucleotide and protein sequence databases”, in: TRENDS in Biochemical Sciences, Vol. 26, 1, 2001, 54-61. After an ESI (ElectroSprayIonization), the trypsin-digested samples were subjected to tandem mass spectrometry. A Q-TOF mass spectrometer having a so-called nanoflow-Z-Spray ion source from Micromass, UK, was used. The procedure corresponded to the working instructions of the equipment manufacturer.

### 3. Identification of GNAT

As shown in FIGS. 1(A) and 1(B), liver cell extracts of baboons to which an LPS injection had been administered contained, inter alia, a novel protein for which a molecular weight of about 37000±700 Dalton was estimated on the basis of the gel electrophoresis data in comparison with marker substances of known molecular weight, while an isoelectric point of about 7.0 to 8.0 was estimated from the relative position of the protein from the first dimension.

This protein was subjected to- a sequence analysis of its N-terminus, as described above. Furthermore, it was analyzed by mass spectrometry, FIG. 2 showing the mass spectrum of the total trypsin-digested protein.

Fragments of the “parent spectrum” according to FIG. 2 were identified by tandem mass spectroscopy. The mass spectrum obtained for these fragments could be identified in a manner known per se computationally as partial peptide sequences (not shown) which had no direct correspondence in the human genome but, on the basis of an existing partial sequence identity, could be assigned to certain segments of the human GNAT according to SEQ ID NO:2.

The partial sequence according to SEQ ID NO:1, identified by sequencing of the N-terminus, was then compared with protein sequences which were available in sequence databases. By means of a program FASTA3 (http://www.ebi.ac.uk/fasta3/), the sequence SEQ ID NO:1 was found as entry 014833 in the “swall” database. The corresponding GenBank entry is AF023466. By means of a program BLAST, the sequence with the GenBank Accession No. AB013093, which on translation gives the amino acid sequence SEQ ID NO:2, was then identified in the database est_human. On the basis of its 76% identity with the known bovine GNAT sequence (GenBank Accession No. AF045032), the amino acid sequence found was identified as that of human GNAT.

The complete sequence of human GNAT, which is thus explicitly known, makes it possible to prepare human GNAT or any partial sequences (fragments) thereof in a controlled manner for human medicine (diagnosis or therapy), and to do so with the use of known synthetic or genetic engineering methods for the preparation of peptides. The same applies to veterinary medicine, it being possible in these cases to rely on corresponding known, e.g. bovine, GNAT sequences, or animal-specific GNAT sequences can readily be found in corresponding animal-specific databases on the basis of the analogies with the known bovine and human sequences. Such peptides can then serve, for example analogously to the procedure described in P. P. Ghillani et al., “Monoclonal antipeptide antibodies as tools to dissect closely related gene products”, The Journal of Immunology, Vol. 141, No. 9, 1998, 3156-3163; and P. P. Ghillani et al., “Identification and Measurement of Calcitonin Precursors in Serum of Patients with Malignant Diseases”, Cancer Research, Vol. 49, No. 23, 1989, 6845-6851, for providing suitable antibodies, in particular monoclonal antibodies, which in turn make it possible to provide assays for the immunodiagnostic determination of GNAT or selected partial peptides thereof.

Monoclonal antibodies obtainable in the known manner described can also serve, particularly after a humanization known per se, for the development of novel therapeutic agents (cf. the therapeutic approaches summarized in K. Garber, “Protein C may be sepsis solution”, Nature Biotechnology, Vol. 18, 2000, 917-918). Furthermore, an in vivo neutralization of GNAT is also permitted by the blockage of the expression of the GNAT gene. The therapeutic interventions resulting from the discoveries in the present Application, also include the administration of active substances which inhibit the enzymatic activity of GNAT.

It is furthermore within the scope of the present invention to use GNAT itself or partial peptides thereof as pharmaceutical active substances. The invention therefore also relates to pharmaceutical compositions which contain, as the actual active substance, one of the peptides according to the invention or antibodies produced against these peptides and prepared for administration to patients, together with a suitable pharmaceutical carrier.

1. Use of glycine N-acyltransferase (GNAT) from body fluids or body tissues in human and veterinary medicine as a marker peptide for diagnosis, for prognosis and for monitoring the course of inflammations and infections and/or as a target for therapeutically influencing the course of inflammations and/or infections.

2. Use of GNAT according to claim 1 in early differential diagnosis and detection, for prognosis, for assessing the severity and for the therapy-accompanying assessment of the course of sepsis and severe infections, in particular sepsis-like systemic infections, by determination of the occurrence and/or the amount of GNAT in a biological fluid or a tissue sample of a patient.

3. Method for early differential diagnosis and detection, for prognosis and assessment of the severity and for the therapy-accompanying assessment of the course of sepsis and severe infections, in particular sepsis-like systemic infections, characterized in that the presence and/or the amount of GNAT in a biological fluid or a tissue sample of a patient is determined and conclusions are drawn with regard to the presence, the expected course, the severity or the success of a treatment of the sepsis or of infection from the presence and/or amount of the fragment determined.

4. Method according to claim 3, characterized in that it is an immunodiagnostic assay method.

5. Method according to claim 3, characterized in that the determination of GNAT is effected indirectly as a determination of the associated GNAT-mRNA or of the GNAT enzymatic activity.

6. Method according to claim 3, characterized in that it is carried out in the course of a multiparameter determination in which simultaneously at least one further sepsis parameter is determined and a measured result is obtained in the form of a set of at least two measured variables which is evaluated for fine diagnosis of sepsis.

7. Method according to claim 6, characterized in that, in the course of multiparameter determination, at least one further parameter which is selected from the group consisting of procalcitonin, CA 19-9, CA 125, S100B, S100A proteins, soluble cytokeratin fragments, in particular CYFRA 21, TPS and/or soluble cytokeratin-1 fragments (sCYIF), the peptides inflammin and CHP, peptide prohormones and the C-reactive protein (CRP) is determined in addition to GNAT.

8. Method according to claim 6, characterized in that the multiparameter determination is carried out as a simultaneous determination by means of a chip technology measuring apparatus or an immunochromatographic measuring apparatus.

9. Method according to claim 8, characterized in that the evaluation of the complex measured result obtained using the measuring apparatus is effected with the aid of a computer program.

10. Method according to claim 7, characterized in that the multiparameter determination is carried out as a simultaneous determination by means of a chip technology measuring apparatus or an immunochromatographic measuring apparatus.


SEQUENCE LISTING (2 sequences)

SEQ ID NO: 1 - protein, 8 aa, Papio sp.
MLPLQGAQ

SEQ ID NO: 2 - protein, 296 aa, Homo sapiens
MMLPLQGAQMLQMLEKSLRKSLPASLKVYGTVFHINHGNPFNLKAVVDKWPDFNTVVVCPQEQDMTDDLDHYTNTYQIYSKDPQNCQEFLGSPELINWKQHLQIQSSQPSLNEAIQNLAAIKSFKVKQTQRILYMAAETAKELTPFLLKSKILSPSGGKPKAINQEMFKLSSMDVTHAHLVNKFWHFGGNERSQRFIERCIQTFPTCCLLGPEGTPVCWDLMDQTGEMRMAGTLPEYRLHGLVTYVIYSHAQKLGKLGFPVYSHVDYSNEAMQKMSYTLQHVPIPRSWNQWNCVPL